(19) United States
(12) Patent Application Publication
(10) Pub. No.: US 2025/0134904 A1
(43) Pub. Date: May 1, 2025

(54) SOLID PHARMACEUTICAL FORMULATIONS OF VARENICLINE

(71) Applicant: MEDICHEM, S.A., SANT JOAN DESPÍ (ES)

(72) Inventors: ISABEL FERNÁNDEZ GARROSA, ÁVILA (ES); MARC SUÑÉ POU, BARCELONA (ES); ERNESTO DURÁN LÓPEZ, CASTELLBISBAL (ES); JOSÉ ANDRÉS LOZANO VELILLA, HOSPITALET DE LLOBREGAT (ES); JORDI PUIG SERRANO, CANET D'ADRI (ES)

(21) Appl. No.: 18/723,102

(22) PCT Filed: Oct. 27, 2022
(86) PCT No.: PCT/EP2022/080147

§ 371 Date: Jun. 21, 2024

(30) Foreign Application Priority Data
Dec. 23, 2021 (EP) 21383207.4

(51) Int. Cl. A61K 31/55; A61K 9/20

(52) U.S. Cl. CPC A61K 31/55; A61K 9/2009; A61K 9/2013; A61K 9/2054

(57) ABSTRACT

Solid pharmaceutical formulations including varenicline or a pharmaceutical salt thereof, and at least a scavenger agent of nitrite anion.

This application claims the benefit of European Patent Application EP21383207.4 filed on 23 Dec. 2021.

### TECHNICAL FIELD

The present invention relates to solid pharmaceutical formulations comprising varenicline or a pharmaceutical salt thereof, and at least a scavenger agent of nitrite anion.

### BACKGROUND ART

Varenicline (a compound represented by formula (I) below) is the international commonly accepted non-proprietary name for 7,8,9,10-tetrahydro-6,10-methano-6H-pyrazino[2,3-h][3]benzazepine (which is also known as 5,8,14-triazatetracyclo[10.3.1.0^{2,11.}0^{4,9}]-hexadeca-2 (11),3,5,7,9-pentaene), and has an empirical formula of C₁₃H₁₃N₃ and a molecular weight of 211.26.

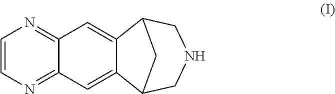 [SMILES: c1cnc2cc3c(cc2n1)C1CNCC3C1]

The L-tartrate salt of varenicline (varenicline L-tartrate) is marketed as film-coated tablets under the name Chantix™ in the United States and Champix™ in the European Union as a smoking cessation treatment. The commercially tablets contain 0.85 mg and 1.71 mg of varenicline tartrate, equivalent to 0.5 mg and 1 mg of varenicline free base, respectively, as active substance, and microcrystalline cellulose, anhydrous calcium hydrogen phosphate, croscarmellose sodium, silica colloidal anhydrous, and magnesium stearate as excipients of the core of the tablet.

Varenicline, and pharmaceutically acceptable acid addition salts thereof, are described in International Publication No. WO9935131, and the L-tartrate salt is specifically described in International Publication No. WO02092089. None of these documents describe a specific solid pharmaceutical formulation of varenicline.

International Publication No. WO03045437 describes varenicline tartrate pharmaceutical tablet compositions manufactured either via a dry granulation process or a direct compression process. Although this document describes stability results for determining the level of total impurities in tablets, it is silent on the identification of these impurities.

International Publication No. WO2004103372 describes formulations of varenicline, or a salt thereof, having less than 4% by weight of the N-formyl varenicline impurity and less than 4% by weight of the N-methyl varenicline impurity that are impurities generated by the reaction of formic acid and/or formaldehyde present in some excipients, with varenicline or a salt thereof.

RD638006A, published in 2017, and RD653031A, published in 2018, describe tablet formulations of the tartrate, fumarate and citrate salts of varenicline showing a satisfactorily stability, i.e., low total impurity formation and no decrease in assay, during storage at accelerated conditions (at 40° C./75% RH) in several different packaging materials (e.g. PVC, PVC/PVC, Triplex or Alu-Alu blister and also HDPE bottles). This document is silent on the identification of these impurities.

Chinese Patent Application No. CN107753449 describes tablets with good content uniformity and stable dissolution containing 0.5-1 mg of varenicline tartrate with D50 of 50-76 microns, 50-70 mg of lactose, 12-26 mg of microcrystalline cellulose, 6-12 mg of calcium hydrogen phosphate, 0.8-1.6 mg of sodium lauryl sulfate and 0.8-1.5 mg of magnesium stearate. This document is silent on the purity and stability of these tablets.

Chinese Patent Applications No. CN109432022 describes pharmaceutical compositions containing varenicline tartrate, a filler, a binder, a disintegrant, a lubricant and a glidant such as silicon dioxide or colloidal silicon dioxide. This document is silent on the purity and stability of these compositions.

Bulletin of the Academy of Sciences of the USSR, Division of chemical science, volume 40, pages 1961-1965 (1991), although relates to the nitrosation of ascorbic acid by nitrite ion in aqueous media, does not mention the use of ascorbic acid in varenicline pharmaceutical formulations.

Journal of Pharmaceutical Sciences, 2021, 110, 3773-3775, describes the use of five inhibitors (ascorbic acid, sodium ascorbate, α-tocopherol, ferulic acid and caffeic acid) for inhibiting nitrosamine formation in solid and in solution/suspension pharmaceutical products containing 4-phenylpiperidine hydrochloride as an active ingredient. However, there are no specific studies to show how these inhibitors could be used in varenicline pharmaceutical products.

N-Nitrosamines (hereinafter referred to simply as “nitrosamines”) are a class of compounds having the chemical structure of a nitroso (—N═O) group bonded to an amine. They can be formed when nitrosating agents (such as nitrite salts under acidic conditions, for example) react with a secondary, a tertiary or a quaternary amine. Nitrosamines are classified as probable human carcinogens on the basis of animal studies.

Nitrites can be found in many excipients at parts per million levels, which typically come from process water, processing steps requiring acid titration, bleaching, and potentially from oxidation in air as the excipient is being heated in a drying process.

Although the US Food and Drug Administration (FDA) has suggested that manufacturers consider using certain antioxidants or excipients, such as sodium carbonate, ascorbic acid (vitamin C) or alpha-tocopherol (vitamin E), to their drug products to inhibit the formation of nitrosamine impurities, there is nothing to suggest that the use of certain antioxidants or excipients can reduce significative the nitrosamines impurities in solid pharmaceutical compositions comprising varenicline or a pharmaceutical salt thereof.

The inhibitory effect that ascorbic acid has on nitrosamine formation was noted as far back as 1976, when investigators proposed that the finished dose form of “easily and rapidly nitrosatable drugs” should include ascorbic acid to prevent the nitrosation of these amine-containing drugs under the strong acidic pH values occurring in the stomach.

The European Medicines Agency (EMA) and the US Food and Drug Administration (FDA) have evaluated the risk of nitrosamine formation or presence during the manufacture of pharmaceutical products and provide guidance to marketing authorization holders to identify and, if necessary, mitigate the risk of presence of nitrosamine impurities. However, there is no consistent approach among the different nitrosamine guidelines regarding the limit for nitrosamines with no available toxicological data. While the current EMA regulatory guidance proposes a default class-specific TTC of 18 ng/day (acceptable daily intake (ADI)) for this nitrosamines, the FDA proposes to use the limit of 26.5 ng/day (acceptable intake of the most potent nitrosamine). In any case, both require the control of nitrosamine impurities at low levels.

Varenicline contains a secondary N, N-dialkyl amine moiety in its structure and therefore can potentially transform into N-nitroso-varenicline impurity (II) under nitrosation conditions. The FDA has established that consuming up to 37 ng/day of N-nitroso-varenicline impurity (II) is considered reasonably safe for humans, based on lifetime exposure. This means that contents of N-nitroso-varenicline impurity (II) must be controlled to not more than 18.5 ppm with respect to varenicline, which is equivalent to not more than 10.82 ppm with respect to varenicline tartrate.

Therefore, there is a clear need for an improved solid pharmaceutical formulation of varenicline with reduced levels of N-nitrosamine impurities, particularly N-nitroso-varenicline impurity (II), stable for a period that is Iong enough to be commercially viable. There is also a need for processes for the obtention of this formulation.

### SUMMARY OF THE INVENTION

In light of the prior art, the technical problem underlying the invention was the provision of an improved solid pharmaceutical composition comprising varenicline, or a pharmaceutical acceptable salt, that limits the formation of nitrosamine impurities.

The inventors have found that solid pharmaceutical formulations of varenicline, or a pharmaceutical acceptable salt thereof, comprising at least a scavenger agent of nitrite anion, reduce the level of nitrosamine impurities to a level significantly lower than the acceptable intakes defined in the guides and are also stable on Iong term storage. Particularly, the solid pharmaceutical formulations of varenicline, or a pharmaceutical acceptable salt thereof, comprising at least a scavenger agent of nitrite anion, limits the formation of N-nitroso-varenicline impurity (II), as defined herein, below its established limit during its manufacturing process and also during Iong term storage.

A first aspect of the invention relates to a solid pharmaceutical composition comprising:
i) varenicline, or a pharmaceutical acceptable salt,
ii) at least a scavenger agent of nitrite anion, and
iii) one or more pharmaceutically acceptable excipients or carriers,
   wherein said composition has not more than 18.5 ppm of N-nitroso-varenicline impurity (II) having the formula below, with respect to varenicline.

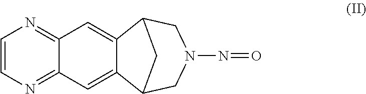 [SMILES: O=NN1CC2CC(C1)c1cc3nccnc3cc12]

Finally, the second aspect of the invention relates to a solid pharmaceutical composition as defined herein above and below, for use as a medicament such as an aid to smoking cessation treatment.

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly through-out the specification and claims.

For the purposes of the present invention, any ranges given include both the lower and the upper end-points of the range. Ranges and values given, such as temperatures, times, and the like, should be considered approximate, unless specifically stated.

The term “Iong term storage” refers to at least 6 months at 40° C. and 75% RH or at least 2 years at 25° C. and 60% RH. Alternatively, it also refers to at least 6 days at 70° C. and 75% RH.

It is noted that, as used in this specification and the appended claims, the singular forms “a”, “an”, and “the” include plural referents unless the context clearly dictates otherwise.

As mentioned above, a first aspect of the invention relates to solid pharmaceutical composition comprising:
i) varenicline, or a pharmaceutical acceptable salt,
ii) at least a scavenger agent of nitrite anion, and
iii) one or more pharmaceutically acceptable excipients or carriers,
   wherein said composition has not more than 18.5 ppm of N-nitroso-varenicline impurity (II) having the formula below, with respect to varenicline.

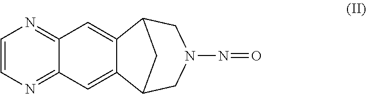 [SMILES: O=NN1CC2CC(C1)c1cc3nccnc3cc12]

The N-nitroso-varenicline-impurity (II) level in the solid pharmaceutical formulation of the present invention may be detected by high performance liquid chromatography (HPLC) method disclosed in the present invention.

For the present invention, the varenicline may be used as free base or in the form of its pharmaceutically acceptable salt. The term “pharmaceutically acceptable salt” refers to non-toxic acid addition salts derived from inorganic and organic acids. Suitable salt derivatives include halides, thiocyanates, sulfates, bisulfates, sulfites, bisulfites, arylsulfonates, alkylsulfates, phosphonates, monohydrogen-phosphates, dihydrogenphosphates, metaphosphates, pyrophosphonates, alkanoates, cycloalkylalkanoates, arylalkonates, adipates, alginates, aspartates, benzoates, fumarates, glucoheptanoates, glycerophosphates, lactates, maleates, nicotinates, oxalates, palmitates, pectinates, picrates, pivalates, succinates, tartrates, citrates, camphorates, camphorsulfonates, digluconates, trifluoroacetates, salicylates, and the like. A particularly preferred salt for the solid pharmaceutical composition of the present invention is the tartrate salt.

Varenicline or a pharmaceutically acceptable salt thereof, preferably varenicline tartrate, can be obtained by any of the processes disclosed in the prior art. Varenicline or a pharmaceutically acceptable salt thereof, preferably varenicline tartrate, can be in any crystalline form, solvate or hydrate, thereof. These forms may differ in some physical properties, but they are equivalent for the purposes of the present invention.

Varenicline or a pharmaceutically acceptable salt thereof, preferably varenicline tartrate, used in the present invention can be subjected to mechanical size reduction, such as micronization, milling or any method known in the art for example cutting, chipping, grinding, crushing, trituration and the like. The forms thus obtained may differ in some physical properties, but they are equivalent for the purposes of the present invention.

The term “scavenger agent of nitrite anion” as used herein, refers to a compound that does not contain any secondary, tertiary or quaternary amine moiety in its structure but is able to react with nitrite anion, thus reducing the available concentration of nitrite anion in the solid pharmaceutical composition and consequently the rate of formation of any potential nitrosamine by-product.

Scavenger agent of nitrite anion for use in the present invention is selected from the group consisting of ascorbic acid, sodium ascorbate, caffeic acid, ferulic acid, and an ammonium salt such as ammonium chloride. A particularly preferred scavenger agent of nitrite anion is ascorbic acid, particularly L-ascorbic acid, also known as vitamin C.

It is known that nitrite anion is not normally a nitrosating agent under neutral to basic conditions (see Ashworth et al. in Org. Process Res. Dev. 2020, 24, 1629-1646). Under strong acidic conditions, nitrite anion transforms into nitrous acid which is the most used nitrosating agent in chemical nitrosation reactions. Nitrous acid is a weak acid with a pKₐ of 3.15. At pH values above its pKₐ, rate of the nitrosation reaction is known to decrease significantly. This decrease in rate with increasing pH arises from the decreases in the concentrations of nitrous acid and H⁺ as the pH is increased, which outweigh the increase in the concentration of unprotonated amine. Reaction rates are also expected to decrease significantly in solid state, due to the reduced mobility of the reacting species.

Nevertheless, reaction in solid state between nitrite anion and 4-phenylpiperidine hydrochloride has been described recently (J. Pharm. Sci. 2021, 110, 3773-3775). This article describes the formation of the corresponding nitrosamine by-product after manufacturing tablets made with 10% (w/w) load of 4-phenylpiperidine hydrochloride as an active ingredient and common excipients which are known to carry nitrite anion as impurities, and stressing the resulting tablets at 50° C./75% RH for one month. Formation of the corresponding nitrosamine impurity can be inhibited significantly by incorporating ascorbic acid, sodium ascorbate or caffeic acid at 1.0% wt % concentration with respect to the total weight of the solid pharmaceutical composition, i.e., 10% with respect to the active ingredient content (wt %) in the solid pharmaceutical composition.

However, the formation of N-nitroso-varenicline impurity (II) in solid pharmaceutical compositions comprising varenicline, or a pharmaceutically acceptable salt thereof, preferably varenicline tartrate, has been found to take place at significant rates even in the presence of basic excipients such as sodium croscarmellose and calcium hydrogen phosphate which should decrease the rate of transformation of nitrite anion into the active nitrosating species, nitrous acid, and even at room temperature. Moreover, it has been found that significantly higher amounts of scavenger agent of nitrite anion than those disclosed in the prior art are needed to prevent the formation of unacceptable contents of N-nitroso-varenicline impurity (II) in solid pharmaceutical compositions comprising varenicline, or a pharmaceutically acceptable salt thereof, preferably varenicline tartrate.

The scavenger agent of nitrite anion is present in the solid pharmaceutical composition of the present invention in an amount between 0.5% w/w and 20% w/w, preferably between 1% and 10%, more preferably between 1.5% and 5% w/w, wherein the percentages are expressed with respect to the total weight of the solid pharmaceutical composition.

The term “about” as used herein refers to a range of values ±10% of a specified value. For example, the expression “about 10” or “around 10” includes ±10% of 10, i.e., from 9 to 11.

The inventors of the present invention have found that by including specific amounts of the scavenger agent of nitrite anion, a solid pharmaceutical composition is provided that complies with the nitrosamines content standards prescribed in the guides of the different regulatory authorities. Particularly, contents of N-nitroso-varenicline impurity (II) as defined herein can be controlled to not more than 18.5 ppm with respect to varenicline.

The expression “pharmaceutically acceptable excipient” refers to excipients that are compatible with the other ingredients of the pharmaceutical composition and are suitable for use in contact with the tissue or organ of humans without excessive toxicity, irritation, allergic response or other complications commensurate with a reasonable benefit/risk ratio.

The pharmaceutically acceptable excipient present in the pharmaceutical composition of the present disclosure is selected from the group comprising of fillers/diluents, disintegrants, lubricants, glidants, and mixtures thereof.

Non-limiting examples of fillers/diluents which can be used in the formulation of the present invention are cellulose derivatives such as microcrystalline cellulose (e.g. Avicel® PH102 or Vivapur® 102), starches (such as maize starch, potato starch, rice starch, wheat starch, pregelatinized starch), lactose, mannitol, sugar and the like, carmellose, sugar alcohols (such as mannitol, sorbitol and xylitol), calcium carbonate, magnesium carbonate, calcium hydrogen phosphate, tribasic calcium phosphate and combinations or mixtures thereof. Preferably, the pharmaceutical composition of the present invention comprises microcrystalline cellulose (e.g., Vivapur® 102) and anhydrous calcium hydrogen phosphate (e.g., Emcompress® Anhydrous).

The total amount of filler/diluent in the solid pharmaceutical of the present is between 30% w/w and 98% w/w, preferably between 75% w/w and 98% w/w, wherein the percentages are expressed with respect to the total weight of the solid pharmaceutical composition.

Non-limiting examples of disintegrants which can be used in the formulation of the present invention are low-substituted hydroxypropyl cellulose, carboxymethylcellulose calcium, carboxymethylcellulose sodium, cellulose powdered, chitosan, docusate sodium, glycine, sodium alginate, croscarmellose sodium, crospovidone and the like, sodium starch glycolate, starch, alginic acid, calcium alginate, and combinations or mixtures thereof. Preferably, the pharmaceutical composition of the present invention comprises croscarmellose sodium (e.g., Vivasol® GF).

The disintegrant is present in the formulation of the present invention at an amount between 0.1% w/w and 5% w/w, more preferably between 0.3% w/w and 2% w/w, and most preferably about 0.5% w/w, wherein the percentages are expressed with respect to the total weight of the solid pharmaceutical composition.

Non-limiting examples of lubricants which can be used in the formulation of the present invention are stearic acid and stearic acid derivatives such as magnesium stearate, calcium stearate, zinc stearate, sucrose esters of fatty acid, polyethylene glycol, talc, sodium stearyl fumarate, sodium lauryl sulfate, zinc stearate, castor oils, waxes and combinations or mixtures thereof. Preferably, the pharmaceutical composition of the present invention comprises magnesium stearate (e.g., Ligamed® MF-2-V).

The lubricant is present in the pharmaceutical composition of the present invention at an amount between 0.5% w/w and 5% w/w, preferably between 1% w/w and 3% w/w, and more preferably about 2%, wherein the percentages are expressed with respect to the total weight of the solid pharmaceutical composition.

Non-limiting examples of glidants which can be used in the formulation of the present invention are colloidal silicon dioxide, talc, fumed silica, starch, starch derivatives, calcium phosphate tribasic, cellulose powdered, magnesium oxide, magnesium silicate, magnesium trisilicate, and bentonite, and combinations or mixtures thereof. Preferably, the formulation of the present invention comprises anhydrous colloidal silicon dioxide (e.g., Aerosil® 200).

The glidant is present in the pharmaceutical composition of the present invention at an amount between 0.1% w/w and 5% w/w, preferably between 0.5% w/w and 3% w/w, and more preferably about 1% w/w, wherein the percentages are expressed with respect to the total weight of the solid pharmaceutical composition.

Additionally, the solid pharmaceutical compositions of the present invention may further include other excipients, such as surfactants and/or binders, as may be desired.

The solid pharmaceutical composition of the present invention can be in form of granules, pellets, tablets and capsules, and the like, preferably, in the form of a tablet.

The solid pharmaceutical composition of the present invention in form of a tablet can be obtained by processes which can involve different techniques used in pharmaceutical development such as direct compression, dry granulation, wet granulation, melt-granulation, spray-drying, extrusion and hot melt extrusion. According to the present invention dry granulation and direct compression are preferred.

Optionally, the solid pharmaceutical composition of the present invention in form of a tablet can be coated and/or packaged in any type of container and/or packaging component that prevents water absorption and degradation (such as bottles, flasks, plastic bags, and blister packs).

The solid pharmaceutical compositions of the present invention can be used to produce tablets, preferably film-coated tablets, containing 0.5 mg and 1 mg of varenicline per tablet. Particularly, tablets containing 0.85 mg and 1.71 mg of varenicline tartrate, equivalent to 0.5 mg and 1 mg of varenicline free base, respectively.

The solid pharmaceutical compositions of the present invention are useful as an aid to smoking cessation.

### EXAMPLES

Hereinafter, the present invention is described in more detail and specifically with reference to the Examples, which however are not intended to limit the present invention.

### HPLC Method Used for Analyzing N-Nitroso-Varenicline Impurity (II)

The chromatographic separation was carried out using an Acquity UPLC HSS PFP, 2.1×100 mm, 1.8 μm at 35±2° C.

Mobile phase A: 0.05% (v/v) formic acid solution in water.

Mobile phase A is prepared as follows: Dilute with water, 0.5 mL of formic acid in a 1000 mL volumetric flask.

Mobile phase B: Acetonitrile LC-MS grade.

The chromatograph was programmed as follows: initial 2 min isocratic 90% mobile phase A; 2-11 min linear gradient to 50% mobile phase A; 11-16 min isocratic 50% mobile phase A; 16-18 min linear gradient to 90% mobile phase A; 18-23 min isocratic 90% mobile phase A.

The chromatograph was equipped with equipped with a mass detector. The acquisition was made in Single Ion Monitoring. The ion with m/z 241.0 was monitored at 0.8 points/s. The ionization mode was positive electrospray (ESI+), the source temperature was set at 130° C., desolvation gas flow was at 500 L/h and the capillary voltage was set at 3 kV. The cone voltage was set at 20 V for the ion with m/z 241.0.

The flow rate was 0.40 mL/min.

The Injection volume was 1 μL.

### Solutions

Diluent: acetonitrile (LC-MS grade): mobile phase A (50:50 v/v).

Test solution (approximately 1.50 mg/ml of varenicline): 60 tablets (0.5 mg dose) or 30 tablets (1.0 mg dose) were suspended in 20 mL of diluent, and the resulting mixture was shaked manually until the tablets were disintegrated. The mixture was sonicated for about 10 minutes and an aliquot was centrifugated for about 10 minutes at 5,000 rpm. The supernatant solution was filtered through a 0.20 μm PVDF filter.

Stock solution of N-nitroso-varenicline impurity (II): About 15.0 mg of N-nitroso-varenicline impurity (II) reference standard were weighted accurately in a 50.0 mL volumetric flask, dissolved, and diluted to volume with acetonitrile (LC-MS grade).

Standard solution of N-nitroso-varenicline impurity (II) at approximately 20.0 ppm with respect to varenicline: 1.0 mL of Stock solution of N-nitroso-varenicline impurity (II) was diluted to 100.0 mL with Diluent. 1.0 mL of the resulting solution was diluted to 100.0 ml with Diluent, to obtain a solution containing 0.03 μg/mL of N-nitroso-varenicline impurity (II) corresponding to approximately 20.0 ppm referred to Test solution.

### Calculation

The quantification (μg/g or ppm) of N-nitroso-varenicline impurity (II) in test specimen was performed as follows:

$$Content ( ppm ) = \frac{{A}_{i}}{{A}_{s}} \times \frac{{C}_{s}}{{C}_{u}}$$

Aᵢ: Area response of N-nitroso-varenicline impurity (II) in Test solution
Aₛ: Area response of N-nitroso-varenicline impurity (II) in Standard solution
Cₛ: Concentration (μg/mL) of N-nitroso-varenicline impurity (II) in Standard solution
Cᵤ: Nominal concentration (g/mL) of varenicline in the Test solution (approximately 0.0015 g/mL).

### General Manufacturing Process 1 (Dry Granulation)

1. Varenicline tartrate, about 17% of the total amount of microcrystalline cellulose (Vivapur®) 102 or Avicel® PH102) and the first load amount of the scavenger agent of nitrite anion were sieved through a 0.8 mm mesh.
2. The sieved components obtained in step 1 were transferred into a drum blender and blended for 10 minutes at between 10 to 15 rpm.
3. Sodium croscarmellose (Vivasol® GF), the remaining amount (about 83%) of microcrystalline cellulose (Vivapur® 102 or Avicel® PH102), anhydrous calcium hydrogen phosphate (Emcompress® anhydrous) and the second load amount of the scavenger agent of nitrite anion, were sieved through a 0.8 mm mesh.
4. The components from step 3 and the blend from step 2 were added into a drum blender and blended for 15 minutes at between 10 to 15 rpm.
5. The resultant blend of step 4 was transferred to a roller compacter to obtain granules using the following parameters:

| Process Parameters | Values | |
|---|---|---|
| Equipment | Alexanderwerk WP120 Pharma | |
| Roller diameter | 120 | mm |
| Roller width | 25 | mm |
| Roller surface | Squared | |
| Vacuum | ON | |
| Screw speed | 40 | rpm |
| Roller speed | 3.4-6 | rpm |
| Roller gap | 1.0 | mm |
| Roller gap control | ON (automatic) | |
| Roller pressure | 45-85 | Bar |
| Pre-granulator | 1.6 | mm |
| Pre-granulator speed | Automatic | |
| Fine-granulator | 0.8 | mm |
| Fine-granulator speed | 90 | rpm |

6. Anhydrous colloidal silicon dioxide (Aerosil® 200) and magnesium stearate (Ligamed® MF-2-V) were sieved through a 0.6 mm mesh and then blended with the granules obtained in step 5 for 2 minutes at between 10 to 15 rpm.
7. The mixture obtained in step 6 was compressed to obtain core tablets using the following parameters:

| Process parameters | Strength 0.5 mg/tablet | Strength 1.0 mg/tablet |
|---|---|---|
| Equipment | Killian 180× tableting | Killian 180× tableting |
| | machine or equivalent | machine or equivalent |
| Compression | 8.0 × 4.0 mm, capsule | 10.0 × 5.0 mm, capsule |
| tooling | shape and biconvex | shape and biconvex |
| | punches, debossed with | punches, debossed with |
| | “0.5” on one face and | “1.0” on one face and |
| | plain on the other face | plain on the other face |
| Wall dimension | 1.1-1.4 | 1.5-1.9 |
| (mm) | | |
| Fill depth (mm) | 4.5-5.2 | 5.8-6.0 |
| Speed (tablets/h) | 9,000 | 9,000 |
| Main compression | 6.9-9.4 | 10.8-11.3 |
| force (kN) | | |

8. The core tablets obtained in step 7, were coated in a Glatt coater using a coating suspension of hydroxypropylmethylcellulose (Opadry® White) in water (15% w/w) at a temperature between 40° C. to 50° C., until reaching a weight increase of about 2.5% with respect to the core tablet weight.

### General Manufacturing Process 2 (Direct Compression)

1. Varenicline tartrate, about 17% of the total amount of microcrystalline cellulose (Vivapur®) 102 or Avicel® PH102) and the scavenger agent of nitrite anion were sieved through a 0.8 mm mesh.
2. The sieved components obtained in step 1 were transferred into a drum blender and blended for 10 minutes at between 10 to 15 rpm.
3. Sodium croscarmellose (Vivasol® GF), the remaining amount (about 83%) of microcrystalline cellulose (Vivapur® 102 or Avicel® PH102) and anhydrous calcium hydrogen phosphate (Emcompress® anhydrous) were sieved through a 0.8 mm mesh.
4. The components from step 3 and the blend from step 2 were added into a drum blender and blended for 15 minutes at between 10 to 15 rpm.
5. Anhydrous colloidal silicon dioxide (Aerosil® 200) and magnesium stearate (Ligamed® MF-2-V) were sieved through a 0.6 mm mesh and then blended with the blend from step 4 for 2 minutes at between 10 to 15 rpm.
6. The mixture obtained in step 5 was compressed to obtain core tablets using the following parameters:

| Process parameters | Strength 0.5 mg/tablet |
|---|---|
| Equipment | Killian 180× tableting machine |
| | or equivalent |
| Compression tooling | 8.0 × 4.0 mm, capsule shape |
| | and biconvex punches, |
| | debossed with “0.5” on one |
| | face and plain on the other |
| | face |
| Wall dimension (mm) | 1.1-1.4 |
| Fill depth (mm) | 4.5-5.2 |
| Speed (tablets/h) | 9,000 |
| Main compression force | 6.9-9.4 |
| (kN) | |

7. The core tablets obtained in step 6, were coated in a Glatt coater using a coating suspension of hydroxypropylmethylcellulose (Opadry® White) in water (15% w/w) at a temperature between 40° C. to 50° C., until reaching a weight increase of about 2.5% with respect to the core tablet weight.

### General Manufacturing Process 3 (Wet Granulation)

1. The scavenger agent of nitrite anion was dissolved in 200 g of purified water.
2. Microcrystalline cellulose (Avicel® PH102), anhydrous calcium hydrogen phosphate (Emcompress® anhydrous) and sodium croscarmellose (Vivasol® GF) were introduced in a high shear mixer and stirred at between 70 to 80 rpm for 10 minutes.
3. The solution obtained in step 1 was added over the mixture obtained in step 2 in about 7.5 minutes, with the high shear mixer operating at between 100 to 125 rpm (impeller) and between 300 to 325 rpm (chopper).
4. The mixture obtained in step 3 was sieved through a 4 mm mesh.
5. The mixture obtained in step 4 was introduced into a fluid bed drier (air inlet temperature 45° C.; air flow 0.9-1.3 m³/h) and dried at 40° C. for about 15 minutes until a final humidity value of about 1.5%.
6. The dried mixture obtained in step 5 was sieved through a 0.8 mm mesh.
7. Varenicline tartrate was sieved through a 0.8 mm mesh.
8. The sieved mixture obtained in step 6 and the sieved component obtained in step 7 were transferred into a drum blended and blended for 10 minutes at between 10 to 15 rpm.
9. Anhydrous colloidal silicon dioxide (Aerosil® 200) and magnesium stearate (Ligamed® MF-2-V) were sieved through a 0.6 mm mesh.
10. The sieved components obtained in step 9 were added to the drum blended containing the blended mixture obtained in step 8, and the resulting mixture was blended for 2 minutes at between 10 to 15 rpm.
11. The mixture obtained in step 10 was compressed to obtain core tablets using the following parameters:

| Process parameters | Strength 0.5 mg/tablet |
|---|---|
| Equipment | Killian 180× tableting machine |
| | or equivalent |
| Compression tooling | 8.0 × 4.0 mm, capsule shape |
| | and biconvex punches, |
| | debossed with “0.5” on one |
| | face and plain on the other |
| | face |
| Wall dimension (mm) | 1.3-1.5 |
| Fill depth (mm) | 5.0-5.2 |
| Speed (tablets/h) | 9,000 |
| Main compression force | 3.0-3.5 |
| (kN) | |

12. The core tablets obtained in step 11, were coated in a Glatt coater using a coating suspension of hydroxypropylmethylcellulose (Opadry White) in water (15% w/w) at a temperature between 40 to 50° C., until reaching a weight increase of about 2.5% with respect to the core tablet weight.

### General Manufacturing Process 4 (Dry Granulation)

1. The total amount of the scavenger agent of nitrite anion was sieved through a 0.250 mesh.
2. Varenicline tartrate, about 17% of the total amount of microcrystalline cellulose (Vivapur® 102 or Avicel® PH102) and about 50% of the total amount of the scavenger agent of nitrite anion (step 1) were sieved through a 0.8 mm mesh.
3. The sieved components obtained in step 2 were transferred into a drum blender and blended for 10 minutes at between 10 to 15 rpm.
4. Sodium croscarmellose (Vivasol® GF), the remaining amount (about 83%) of microcrystalline cellulose (Vivapur® 102 or Avicel® PH102), anhydrous calcium hydrogen phosphate (Emcompress® anhydrous) and the second load amount of the scavenger agent of nitrite anion (step 1), were sieved through a 0.8 mm mesh.
5. The components from step 4 and the blend from step 3 were added into a drum blender and blended for 15 minutes at between 10 to 15 rpm.
6. The resultant blend of step 5 was transferred to a roller compacter to obtain granules using the following parameters:

| Process Parameters | Values | |
|---|---|---|
| Equipment | Alexanderwerk WP200 Pharma | |
| Roller diameter | 200 | mm |
| Roller width | 75 | mm |
| Roller surface | Grooved and knurled | |
| Vacuum | ON | |
| Screw speed | 30 | rpm |
| Roller speed | 8.8-10 | rpm |
| Roller gap | 1.5 | mm |
| Roller gap control | ON (automatic) | |
| Roller pressure | 45-100 | Bar |
| Pre-granulator | 1.6 | mm |
| Pre-granulator speed | 70 | rpm |
| Fine-granulator | 0.8 | mm |
| Fine-granulator speed | 90 | rpm |

7. Anhydrous colloidal silicon dioxide (Aerosil® 200) is blended with the granules obtained in step 6 for 5 minutes at between 10 to 15 rpm. The mixture obtained was sieved through a 0.8 mm mesh.
8. Magnesium stearate (Ligamed® MF-2-V) was sieved through a 0.8 mm mesh and then blended with the blend obtained in step 7 for 2 minutes at between 10 to 15 rpm.
9. The mixture obtained in step 8 was compressed to obtain core tablets using the following parameters:

| Process parameters | Strength 0.5 mg/tablet | Strength 1.0 mg/tablet |
|---|---|---|
| Equipment | Kilian Tablet Press E150 | Kilian Tablet Press E150 |
| | Plus tableting machine or | Plus tableting machine or |
| | equivalent | equivalent |
| Compression | 8.0 × 4.0 mm, capsule | 10.0 × 5.0 mm, capsule |
| tooling | shape and biconvex | shape and biconvex |
| | punches, debossed with | punches, debossed with |
| | “0.5” on one face and | “1.0” on one face and |
| | plain on the other face | plain on the other face |
| Web Heigh Dial | 1.0 | 1.4 |
| (mm) | | |
| Total Fill (mm) | 4.4 | 6.6 |
| Speed (tablets/h) | 60,000 | 60,000 |
| Main compression | 8.5-10.2 | 14.0-14.5 |
| force(kN) | | |

10. The core tablets obtained in step 9, were coated in a Glatt coater using a coating suspension of hydroxypropylmethylcellulose (Opadry® White) in water (15% w/w) at a temperature between 40° C. to 50° C., until reaching a weight increase of about 3% with respect to the core tablet weight.

### Comparative Examples 1 to 2

The following composition tablets were obtained using the above general manufacturing processes. The varenicline tartrate used as starting material was found to contain less than 0.4 ppm of N-nitroso-varenicline impurity (II).

Amounts of the different ingredients used for the manufacture of these comparative examples are disclosed in the table below.

| | | | N-nitroso-varenicline impurity | |
|---|---|---|---|---|
| | | | (II) contents (ppm with respect | |
| | | | to varenicline) | |
| | | | | After |
| | | | | 6 months |
| | | | | at 40° C., |
| | General | | | 75% RH in |
| Comparative | manufacturing | | After | PVC/Alu |
| Example | process | Ingredients (mg/tablet) | manufacture | blisters |
| 1 | 1 | Varenicline tartrate (0.88) | 130.80 | 227.61 |
| | | Avicel ® PH102 (46.55) | | |
| | | Emcompress ® anhydrous | | |
| | | (51.00) | | |
| | | Vivasol ® GF (0.51) | | |
| | | Aerosil ® 200 (1.02) | | |
| | | Ligamed ® MF-2-V (2.04) | | |
| | | Scavenger agent of nitrite | | |
| | | anion: None | | |
| 2 | 1 | Varenicline tartrate (1.71) | 111.16 | 270.89 |
| | | Avicel ® PH102 (93.15) | | |
| | | Emcompress ® anhydrous | | |
| | | (102.00) | | |
| | | Vivasol ® GF (1.02) | | |
| | | Aerosil ® 200 (2.04) | | |
| | | Ligamed ® MF-2-V (4.08) | | |
| | | Scavenger agent of nitrite | | |
| | | anion: None | | |

### Comparative Examples 3 to 4

The following composition tablets were obtained using the above general manufacturing processes. The varenicline tartrate used as starting material was found to contain less than 0.4 ppm of N-nitroso-varenicline impurity (II).

Amounts of the different ingredients used for the manufacture of these comparative examples are disclosed in the table below.

| | | | N-nitroso-varenicline impurity | |
|---|---|---|---|---|
| | | | (II) contents (ppm with respect | |
| | | | to varenicline) | |
| | General | | | After 14 days |
| Comparative | manufacturing | | After | at 70° C., |
| Example | process | Ingredients (mg/tablet) | manufacture | 75% RH |
| 3 | 2 | Varenicline tartrate (0.88) | 12.78 | 52.73 |
| | | Vivapur ® 102 (46.55) | | (open dish) |
| | | Emcompress ® anhydrous | | |
| | | (51.00) | | |
| | | Vivasol ® GF (0.51) | | |
| | | Aerosil ® 200 (1.02) | | |
| | | Ligamed ® MF-2-V (2.04) | | |
| | | Scavenger agent of nitrite | | |
| | | anion: None | | |
| 4 | 1 | Varenicline tartrate (0.88) | 6.69 | 108.38 |
| | | Vivapur ® 102 (46.55) | | (blisters |
| | | Emcompress ® anhydrous | | PVC/Alu) |
| | | (51.00) | | |
| | | Vivasol ® GF (0.51) | | |
| | | Aerosil ® 200 (1.02) | | |
| | | Ligamed ® MF-2-V (2.04) | | |
| | | Scavenger agent of nitrite | | |
| | | anion: None | | |

### Examples 1 to 7

The following composition tablets were obtained using the above general manufacturing processes. The varenicline tartrate used as starting material was found to contain less than 0.4 ppm of N-nitroso-varenicline impurity (II).

Amounts of the different ingredients used for the manufacture of these examples are disclosed in the table below.

| | | | N-nitroso-varenicline impurity | |
|---|---|---|---|---|
| | | | (II) contents (ppm with respect | |
| | | | to varenicline) | |
| | General | | | After stress |
| | manufacturing | | After | study at 70° C., |
| Example | process | Ingredients (mg/tablet) | manufacture | 75% RH |
| 1 | 3 | Varenicline tartrate (0.88) | Not | 6.38 |
| | | Avicel ® PH102 (31.25) | measured | (14 days, open dish) |
| | | Emcompress ® anhydrous | | |
| | | (51.00) | | |
| | | Vivasol ® GF (0.51) | | |
| | | Aerosil ® 200 (1.02) | | |
| | | Ligamed ® MF-2-V (2.04) | | |
| | | Ammonium chloride (15.30) | | |
| 2 | 1 | Varenicline tartrate (0.88) | 2.96 | 8.40 |
| | | Vivapur ® 102 (46.04) | | (6 days, open dish) |
| | | Emcompress ® anhydrous | | |
| | | (51.00) | | |
| | | Vivasol ® GF (0.51) | | |
| | | Aerosil ® 200 (1.02) | | |
| | | Ligamed ® MF-2-V (2.04) | | |
| | | Ascorbic acid 1^{st} load (0.51) | | |
| | | Ascorbic acid 2^{nd} load (0.00) | | |
| 3 | 1 | Varenicline tartrate (0.88) | 2.45 | 4.65 |
| | | Vivapur ® 102 (45.53) | | (6 days, open dish) |
| | | Emcompress ® anhydrous | | |
| | | (51.00) | | |
| | | Vivasol ® GF (0.51) | | |
| | | Aerosil ® 200 (1.02) | | |
| | | Ligamed ® MF-2-V (2.04) | | |
| | | Ascorbic acid 1^{st} load (1.02) | | |
| | | Ascorbic acid 2^{nd} load (0.00) | | |
| 4 | 1 | Varenicline tartrate (0.88) | Not | 1.71 |
| | | Vivapur ® 102 (45.02) | measured | (14 days, blisters |
| | | Emcompress ® anhydrous | | PVC/Alu) |
| | | (51.00) | | |
| | | Vivasol ® GF (0.51) | | |
| | | Aerosil ® 200 (1.02) | | |
| | | Ligamed ® MF-2-V (2.04) | | |
| | | Ascorbic acid 1^{st} load (1.53) | | |
| | | Ascorbic acid 2^{nd} load (0.00) | | |
| 5 | 1 | Varenicline tartrate (0.88) | 2.63 | 2.55 |
| | | Vivapur ® 102 (44.00) | | (10 days, open dish) |
| | | Emcompress ® anhydrous | | |
| | | (51.00) | | |
| | | Vivasol ® GF (0.51) | | |
| | | Aerosil ® 200 (1.02) | | |
| | | Ligamed ® MF-2-V (2.04) | | |
| | | Ascorbic acid 1^{st} load (2.55) | | |
| | | Ascorbic acid 2^{nd} load (0.00) | | |
| 6 | 1 | Varenicline tartrate (0.88) | Not | Not detected |
| | | Vivapur ® 102 (41.45) | detected | (14 days, blisters |
| | | Emcompress ® anhydrous | | PVC/Alu) |
| | | (51.00) | | |
| | | Vivasol ® GF (0.51) | | |
| | | Aerosil ® 200 (1.02) | | |
| | | Ligamed ® MF-2-V (2.04) | | |
| | | Ascorbic acid 1^{st} load (2.55) | | |
| | | Ascorbic acid 2^{nd} load (2.55) | | |
| 7 | 2 | Varenicline tartrate (0.88) | 2.07 | 3.10 |
| | | Avicel ® PH102 (41.45) | | (14 days, open dish) |
| | | Emcompress ® anhydrous | | |
| | | (51.00) | | |
| | | Vivasol ® GF (0.51) | | |
| | | Aerosil ® 200 (1.02) | | |
| | | Ligamed ® MF-2-V (2.04) | | |
| | | Ascorbic acid (5.10) | | |

In Example 1, the scavenger agent of nitrite anion (ammonium chloride) represents 15% wt % concentration with respect to the total weight of the solid pharmaceutical composition, i.e., 3,060% with respect to the varenicline weight content in the solid pharmaceutical composition.

In Example 2, the scavenger agent of nitrite anion (ascorbic acid) represents 0.5% wt % concentration with respect to the total weight of the solid pharmaceutical composition, i.e., 102% with respect to the varenicline weight content in the solid pharmaceutical composition.

In Example 3, the scavenger agent of nitrite anion (ascorbic acid) represents 1.0% wt % concentration with respect to the total weight of the solid pharmaceutical composition, i.e., 204% with respect to the varenicline weight content in the solid pharmaceutical composition.

In Example 4, the scavenger agent of nitrite anion (ascorbic acid) represents 1.5% wt % concentration with respect to the total weight of the solid pharmaceutical composition, i.e., 306% with respect to the varenicline weight content in the solid pharmaceutical composition.

In Example 5, the scavenger agent of nitrite anion (ascorbic acid) represents 2.5% wt % concentration with respect to the total weight of the solid pharmaceutical composition, i.e., 501% with respect to the varenicline weight content in the solid pharmaceutical composition.

In Examples 6 and 7, the scavenger agent of nitrite anion (ascorbic acid) represents 5.0% wt % concentration with respect to the total weight of the solid pharmaceutical composition, i.e., 1,020% with respect to the varenicline weight content in the solid pharmaceutical composition.

### Examples 8 to 10

The following composition tablets were obtained using the above general manufacturing processes. The varenicline tartrate used as starting material was found to contain less than 0.4 ppm of N-nitroso-varenicline impurity (II).

Amounts of the different ingredients used for the manufacture of these comparative examples are disclosed in the table below.

| | | | N-nitroso-varenicline impurity | |
|---|---|---|---|---|
| | | | (II) contents (ppm with respect | |
| | | | to varenicline) | |
| | General | | | After 3 months |
| | manufacturing | | After | at 40° C., |
| Example | process | Ingredients (mg/tablet) | manufacture | 75% RH |
| 8 | 1 | Varenicline tartrate (0.88) | Not | Not detected |
| | | Vivapur ® 102 (46.55) | detected | (blister |
| | | Emcompress ® anhydrous | | PVC/Alu) |
| | | (51.00) | | |
| | | Vivasol ® GF (0.51) | | |
| | | Aerosil ® 200 (1.02) | | |
| | | Ligamed ® MF-2-V (2.04) | | |
| | | Ascorbic acid 1^{st} load (2.55) | | |
| | | Ascorbic acid 2^{nd} load (2.55) | | |
| 9 | 4 | Varenicline tartrate (0.88) | Not | 121.23 |
| | | Avicel PH102 (46.55) | measured | (Blister |
| | | Emcompress ® anhydrous | | PVC/Alu) |
| | | (51.00) | | |
| | | Vivasol ® GF (0.51) | | |
| | | Aerosil ® 200 (1.02) | | |
| | | Ligamed ® MF-2-V (2.04) | | |
| | | Scavenger agent of nitrite anion: | | |
| | | None | | |
| 10 | 4 | Varenicline tartrate (0.88) | Not | Not detected |
| | | Vivapur ® 102 (46.55) | detected | (blister |
| | | Emcompress ® anhydrous | | Aclar/Alu) |
| | | (51.00) | | |
| | | Vivasol ® GF (0.51) | | |
| | | Aerosil ® 200 (1.02) | | |
| | | Ligamed ® MF-2-V (2.04) | | |
| | | Ascorbic acid 1^{st} load (2.55) | | |
| | | Ascorbic acid 2^{nd} load (2.55) | | |

What is claimed is:

1. A solid pharmaceutical composition comprising:
i) varenicline, or a pharmaceutical acceptable salt,
ii) at least a scavenger agent of nitrite anion, and
iii) one or more pharmaceutically acceptable excipients or carriers,
wherein said composition has not more than 18.5 ppm of N-nitroso-varenicline impurity (II) having the formula below, with respect to varenicline.

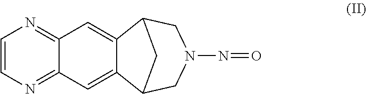 [SMILES: O=NN1CC2CC(C1)c1cc3nccnc3cc12]

2. The solid pharmaceutical composition according to claim 1, wherein the pharmaceutical acceptable salt is varenicline tartrate.

3. The solid pharmaceutical composition according to claim 1, wherein the scavenger agent of nitrite anion is present in an amount between 0.5% w/w and 20% w/w, preferably between 1% w/w and 10% w/w, more preferably between 1.5% w/w and 5% w/w, wherein the percentages are expressed with respect to the total weight of the solid pharmaceutical composition.

4. The solid pharmaceutical composition according to claim 3, wherein the scavenger agent of nitrite anion is selected from the group consisting of ascorbic acid, sodium ascorbate, caffeic acid, ferulic acid, and an ammonium salt such as ammonium chloride.

5. The solid pharmaceutical composition according to claim 4, wherein the scavenger agent of nitrite anion is ascorbic acid, particularly L-ascorbic acid, also known as vitamin C.

6. The solid pharmaceutical composition according to claim 1, wherein one or more pharmaceutically acceptable excipients or carriers are selected from the group comprising of diluent(s), disintegrant(s), glidant(s), lubricant(s), and combinations thereof.

7. The solid pharmaceutical composition according to claim 6, wherein the diluents are microcrystalline cellulose and calcium hydrogen phosphate anhydrous, the disintegrant is croscarmellose sodium, the glidant is anhydrous colloidal silicon dioxide, and the lubricant is magnesium stearate.

8. The solid pharmaceutical composition according to claim 1, wherein said composition is in form of a tablet.

9. (canceled)

10. The solid pharmaceutical composition according to claim 2, wherein the scavenger agent of nitrite anion is present in an amount between 0.5% w/w and 20% w/w, preferably between 1% w/w and 10% w/w, more preferably between 1.5% w/w and 5% w/w, wherein the percentages are expressed with respect to the total weight of the solid pharmaceutical composition.

11. The solid pharmaceutical composition according to claim 10, wherein the scavenger agent of nitrite anion is selected from the group consisting of ascorbic acid, sodium ascorbate, caffeic acid, ferulic acid, and an ammonium salt such as ammonium chloride.

12. The solid pharmaceutical composition according to claim 11, wherein one or more pharmaceutically acceptable excipients or carriers are selected from the group comprising of diluent(s), disintegrant(s), glidant(s), lubricant(s), and combinations thereof.

13. The solid pharmaceutical composition according to claim 12, wherein the diluents are microcrystalline cellulose and calcium hydrogen phosphate anhydrous, the disintegrant is croscarmellose sodium, the glidant is anhydrous colloidal silicon dioxide, and the lubricant is magnesium stearate.

14. The solid pharmaceutical composition according to claim 13, wherein said composition is in form of a tablet.

15. (canceled)

16. A method of smoking cessation comprising administering to a human in need thereof the solid pharmaceutical composition according to claim 1.

17. A method of smoking cessation comprising administering to a human in need thereof the tablet according to claim 8.

18. A method of smoking cessation comprising administering to a human in need thereof the solid pharmaceutical composition according to claim 13.
